(19) United States
(12) Patent Application Publication
(10) Pub. No.: US 2010/0239687 A1
(43) Pub. Date: Sep. 23, 2010

(54) USES OF RARE EARTH ELEMENTS FOR SLIMMING

(75) Inventors: Sang Kook Kim, Daegu (KR); Hyuk Jung, Daegu (KR)

Correspondence Address: CHRISTOPHER PAUL MITCHELL, 888 16TH ST., NW, SUITE 800, WASHINGTON, DC, 20006, US

(73) Assignee: TAEWUNG ELS, INC., (KR)

(21) Appl. No.: 12/599,812

(22) PCT Filed: May 9, 2008
(86) PCT No.: PCT/KR08/02635

§ 371 Date: Nov. 11, 2009

(30) Foreign Application Priority Data
May 11, 2007 (KR) 10-2007-0046248

(51) Int. Cl. A61K 8/19; A61Q 19/00; A61Q 19/06; A61P 3/04; A61K 33/24

(52) U.S. Cl. 424/617

(57) ABSTRACT

Disclosed is a use of rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide, for promoting lipidolysis, anti-cellulite, enhancing skin firmness, or for slimming.

### TECHNICAL FIELD

The present invention relates to a use of rare earth element for slimming. More specifically, it relates to the use of rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof, for promoting lipidolysis, anti-cellulite, enhancing skin firmness, and for slimming.

### BACKGROUND ART

A human body has approximately 20 billion adipocytes. The adipocytes play a role in accumulating or releasing energy in a mammalian body. The adipocytes have a complicated mechanism for regulating accumulation and release of energy. If supplies of energy are extremely excessive to demands thereon, triglycerides are stored in the adipocytes, which are degraded to glycerol and free fatty acids and consumed, upon the exhaustion of energy. Obesity occurs when excessive energy is accumulated due to imbalance of the above process. It generally refers to abnormal hypertrophy of subcutaneous adipose tissue under the skin layer, due to the increased size and number of adipocytes. It usually results from the accumulation of excessive calories in the form of lipid in a body, when calories obtained from food are unnecessarily more than those consumed into energy. Because of the thickness of lipid accumulated under the skin layer, it makes the body fat and eliminates physical beauty, and further, is a cause of various adult diseases. The diseases that are closely associated with obesity include hypertension, arteriosclerosis, cerebrovascular disease, hyperlipidemia, ischemic heart disease, hypoxia, pulmonary hypertension, arthritis, fatty liver, toxemia of pregnancy, diabetes, fatigue, shortness of breath, etc. The increase of adipose tissues in the body lays a burden on the heart to supply blood thereto, which elevates blood pressure. Also, the increase of adipose tissues causes the increase of insulin demand in a body, and so lays an increased burden on the pancreas to produce insulin, ultimately to decrease producibility and secretability of insulin of pancreas in the long term. Therefore, in case of hypertension and diabetes due to obesity, blood pressure can be dropped and blood glucose can be controlled by the loss of weight. In addition, obesity has been reported to increase the frequency of arthritis, gout, respiratory dysfunction, sterility, menstrual irregularity, such cancers as intestinal cancer and breast cancer in women, etc. As such, obesity, as one of major causes to shorten human life, has been reported to cause various adult diseases as well as breast cancer, uterine cancer, colon cancer, etc., and now is considered one of fatal diseases. (J. Biol. Chem., 273, 32487˜32490 (1998) and Nature, 404, 652˜660 (2000)). The obesity spreads world-widely, and further, appears in every age group including childhood, boy- or girlhood, adolescence, and adulthood.

Cellulite describes a condition that the skins become uneven like orange peel, due to circulatory disorders from the accumulation of excessive fat and wastes. It occurs only in female skin and subcutaneous fat. In addition to the abnormal excessive accumulation of subcutaneous adipose tissue, cellulite obesity occurs when lymphatic fluid is backed up in the inter-tissue space due to the abnormality of lymphatic system, and so a kind of edema appears in that region to look like a lump. If lymphatic fluid is backed up in the inter-tissue space, it is not just a matter of beauty like obesity, but toxic substances are accumulated in the body, having adverse effects on skin cells and inhibiting ionization for cell nutrition, thereby causing not only aging of the skin but also necrosis of cells. Therefore, in order to prevent cellulite obesity, it is necessary to drain the backed-up lymphatic fluid. As described above, cellulite has the different cause from general obesity, but is basically caused by the increased size and number of adipocytes. Thus, degradation and excretion of fat in adipocytes can give great help to keep health and to make the body slim and smooth.

Currently, agents for treating obesity are largely classified into those that act in the central nerve system to control appetite and those that act in the gastrointestinal tract to inhibit absorption. Many of the drugs that had been previously used were recently prohibited, since they have shown side effects such as primary pulmonary hypertension and valvular lesion. They also have shown reduced blood pressure and lactic acidosis, and so cannot be used for patients with heart failure and renal disease. Recently, there have been continuously increasing demands on cosmetics for slimming and anti-cellulite effective in eliminating excessive subcutaneous fat and improving skin firmness. The known cosmetics for suppressing and treating obesity comprise caffeine, theophylline, L-carnitine, Centella Asiatica, ruscus, focus, ivy, horse chestnut, and fragrance products containing other inorganic substances. They showed approved, but unsatisfactory, effects. PCT Publication No. WO04/093865 disclosed for the first time that theanine, a taste element of green tea, promotes lipidolysis, similarly to caffeine, which had been known as a positive control of lipidolysis in the cosmetic field, but through a different mechanism therefrom. Also, PCT Publication No. WO04/084885 disclosed that genistein and carnitine can effectively prevent and suppress obesity through a different mechanism from theanine or caffeine.

Rare earth elements are trace metal elements, which are contained at about 0.016% in the earth's crust, and refer to minerals including 17 elements, 15 elements of atomic numbers from 57 to 71 (La, Ce, Pr, Nd, Pm, Sm, Eu, Gd, Tb, Dy, Ho, Er, Tm, Yb, Lu) and 2 elements of group 3A (Sc, Y) in the periodic table of elements. They exist in the form of mixtures of elements sharing very similar physical and chemical properties, and their separation into each single element is complicated, but possible, by using slight differences from one another. Rare earth elements may exist in the form of a salt in which it is ionically bonded with its counter-ion, for example, in the form of sulfate, nitrate, carbonate, acetate, phosphate, chloride, etc., or in the form of oxide including hydroxide. Rare earth elements are very useful even though they were found only two hundred years ago, and are not still well known to public. Since rare earth elements have unique physical and chemical effects due to their unique electronic structures, they have been used in machinery, petrochemistry, photochemistry, etc. as permanent magnet, superconductor or fluorophore, and have been recently tried to be applied in various fields of agriculture, forestry, livestock industry, etc. Furthermore, as biological effects, rare earth elements promote photosynthetic action and chlorophyll production in higher plants; promote shoot formation; increase root vitality; activate respiratory function; promote nutrient transport in plant body; control moisture; promote cell division, hormone transfer, and absorption and transport of nutrients; activates metabolism; increases synthesis of protein and RNA in leaves, etc. In addition, they retard aging of leaves, increase protein contents in green algae, and promote synthesis of protein and chlorophyll by promoting photosynthesis and oxygen releasing activity. La³⁺ is known to increase activity of (Na⁺, K⁺)-ATPase and Mg²⁺-ATPase in cell membrane of human erythrocytes, and thus, strengthen functions of Ca²⁺ ion in human body (The journal of Biological Chemistry, 1986; 261(20); 9552-9557). Some of rare earth elements are known to have antibacterial (Chem. Pharm. Bull, 2003; 51(5); 494-498) and antioxidative activities by preventing generation of reactive oxygen species (Biochemical and Biophysical Research Communication, 2006; 2; 86-91). They are also known to increase weight and feed conversion ratio (FCR) in growing fat pigs (J. Anim. Physiol. a. Anim. Nutr., 2001; 85; 263-270). Furthermore, it was found that rare earth elements are low toxic, absorbed by a very small amount through digestive organs, scarcely accumulated in body, and not teratogenic, mutagenic or carcinogenic (Environmental Health Perspectives, 1996; 104; 85-95). However, it has been neither taught nor suggested that rare earth elements, salts or oxides thereof, or mixtures thereof can show slimming effects by promoting lipidolysis, eliminating cellulite, or enhancing skin firmness.

### DISCLOSURE OF INVENTION

### Technical Subject

In the light of causes of obesity, the present inventors contemplated that reduction of body fat is crucial for resolving obesity, rather than a mere weight loss, and so it would be essential to find out a way to degrade unnecessary fat accumulated in the body and to activate its oxidation. Then, they conducted the extensive studies to find out an ingredient that is safe to the human body and able to promote lipidolysis in adipocytes and to effectively enhance fat oxidation. As a result, they found out that one or more rare earth elements are effective for promoting degradation of neutral fat in adipocytes, and moreover, that a composition containing one or more rare earth elements reduces the thickness of subcutaneous fat and cellulite by excellent lipidolysis-promoting, anti-cellulite, and skin firmness-enhancing activities, thereby to give great help to make and keep the skin and the body slim and firm, and thus, completed the present invention.

Therefore, the first object of the present invention is to provide a composition for promoting lipidolysis, anti-cellulite, enhancing skin firmness, or for slimming, containing one or more rare earth elements as an active ingredient.

The second object of the present invention is to provide a use of one or more rare earth elements for promoting lipidolysis, anti-cellulite, enhancing skin firmness, or for slimming.

The third object of the present invention is to provide a method for promoting lipidolysis, anti-cellulite, enhancing skin firmness, or for slimming, comprising using one or more rare earth elements.

The fourth object of the present invention is to provide a method for preparing an agent for promoting lipidolysis, anti-cellulite, enhancing skin firmness, or for slimming, by using one or more rare earth elements.

### Technical Solution

The first aspect of the present invention relates to a composition containing, as an active ingredient, rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof, for promoting lipidolysis, anti-cellulite, enhancing skin firmness, or for slimming.

The second aspect of the present invention relates to a use of rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof, for promoting lipidolysis, anti-cellulite, enhancing skin firmness, or for slimming.

The third aspect of the present invention relates to a method for promoting lipidolysis, anti-cellulite, enhancing skin firmness, or for slimming in a subject in need thereof, comprising the step of applying to the subject rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof.

The fourth aspect of the present invention relates to a method for preparing an agent for promoting lipidolysis, anti-cellulite, enhancing skin firmness, or for slimming, comprising using rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof.

### BRIEF DESCRIPTION OF DRAWINGS

FIG. 1 is a graph showing the lipidolysis effect of mixed rare earth elements in adipocytes of rats;

FIG. 2 is a graph showing the lipidolysis effect of mixed rare earth elements in 3T3 L1 cell line;

FIG. 3 is a graph showing the effect of the composition of the present invention on skin firmness;

FIG. 4 is a graph showing weight change by oral administration of mixed rare earth elements;

FIG. 5 is a graph showing the lipidolysis effect by oral administration of mixed rare earth elements;

FIG. 6 is a graph showing weight change by oral administration of mixed rare earth elements in high-fat diet maintaining group of obesity-induced animals;

FIG. 7 is a graph showing weight change by oral administration of mixed rare earth elements in normal diet conversion group of obesity-induced animals.

### MODE FOR CARRYING OUT THE INVENTION

Hereinafter, the present invention will be described in detail.

The term ‘slimming’ used in the present invention refers to not only suppression of obesity in view of health but also making the skin firm and smooth by reduction of cellulite.

Rare earth elements share similar physicochemical properties, and kinds of rare earth elements which can be used in the present invention are not specifically limited, and any one of rare earth elements may be used alone or in admixture with other rare earth elements. A salt of rare earth element may also be used as long as it is non-toxic which is pharmaceutically or cosmetically acceptable. Examples thereof include, but are not limited to, sulfate, nitrate, carbonate, acetate, phosphate, chloride, etc. Further, an oxide of rare earth element (for example, Ce₂O₃, La₂O₃, etc.) may also be used without limit as long as it is non-toxic which is pharmaceutically or cosmetically acceptable. In the present invention, the oxide of rare earth element includes hydroxide of rare earth element (for example, Ce(OH)₃, La(OH)₃, etc.).

For use in the present invention, rare earth elements may be pulverized into fine powder, and may be contained in 0.0001 to 20% by weight, particularly 0.001 to 1% by weight.

The composition according to the present invention promotes degradation of neutral fat in cells and selectively eliminates subcutaneous fat, upon spread onto the skin, and thus, helps to make the body slim. Further, the composition reduces not only subcutaneous fat or intra-abdominal fat but also dimples and lumps caused by obese adipocytes in the cellulite region of women, showing excellent anti-cellulite effect to regain skin firmness and smoothness. In particular, the composition of the present invention has no harm on the human body and little skin irritability, and so can be used safely. That is, while the previous agents for improving obesity were passive ones that aimed only at promoting differentiation of adipocytes or lipidolysis, the composition of the present invention is active one that degrades the formed fat in adipocytes as well as completely eliminates the degradation products to prevent the re-accumulation of neutral fat, thereby to resolve and suppress obesity.

In case of manufacturing the composition of the present invention into a cosmetic, it can be manufactured into any formulations that are conventional in the art. Examples thereof include, but are not limited to, solution, suspension, emulsion, paste, gel, cream, lotion, powder, soap, surfactant-containing cleanser, oil, powdered foundation, emulsion foundation, wax foundation, spray, etc. More specifically, it can be manufactured into hydrating toner, nutrition toner, nutrition lotion, massage cream, nutrition cream, serum, eye cream, cleansing cream, cleansing foam, cleansing water, pack, gel, spray, or powder. The cosmetic composition of the present invention may contain conventional ingredients for cosmetics, in addition to the active ingredient. Examples thereof include conventional auxiliaries or carries such as antioxidants, stabilizers, solubilizing agents, vitamins, pigments, and perfumes. If the formulation of the present invention is paste, cream, or gel, animal oil, vegetable oil, wax, paraffin, starch, tragacanth, cellulose derivative, polyethylene glycol, silicone, bentonite, silica, talc, or zinc oxide may be used as carriers. If the formulation of the present invention is powder or spray, lactose, talc, silica, aluminum hydroxide, calcium silicate, or polyamide powder may be used as carriers. Particularly, for spray, propellants such as chlorofluorohydrocarbon, propane/butane or dimethylether can be additionally comprised. When the formulation of the present invention is solution or emulsion, solvents, solubilizing agents, or emulsifying agents, such as water, ethanol, isopropanol, ethyl carbonate, ethyl acetate, benzyl alcohol, benzyl benzoate, propyleneglycol, 1,3-butylglycol oil, glycerol aliphatic ester, polyethyleneglycol, or fatty acid ester of sorbitan may be used as carriers. If the formulation of the present invention is suspension, liquid diluents such as water, ethanol, or propyleneglycol; suspending agents such as ethoxylated isostearyl alcohol, polyoxyethylene sorbitol ester, and polyoxyethylene sorbitan ester; microcrystalline cellulose, aluminum metahydroxide, bentonite, agar, tragacanth, etc. may be used as carriers. If the formulation of the present invention is a surfactant-containing cleanser, the carriers that can be used include aliphatic alcohol sulfate, aliphatic alcohol ether sulfate, sulfosuccinic acid monoester, isethionate, imidazolium derivative, methyltaurate, sarcosinate, fatty acid amide ether sulfate, alkylamidobetaine, aliphatic alcohol, fatty acid glyceride, fatty acid diethanolamide, vegetable oil, lanolin derivative, ethoxylated glycerol fatty acid ester, etc.

In case the composition of the present invention is manufactured into a pharmaceutical formulation, the active ingredient may be combined with one or more pharmaceutically acceptable carriers, and, depending upon the purpose of administration, manufactured into orally administrable formulations such as tablets, hard or soft capsules, granules, chewable tablets, pills, powder, elixir, suspension, emulsion, solution, and syrup, or parenterally administrable formulations such as aerosol, sachet, sterilized injection solution, and sterilized powder. When the active ingredient of the present invention is formulated into tablets, capsules, granules, chewable tablets, pills, powder, elixir, suspension, emulsion, solution, syrup, etc. for the purpose of oral administration, the formulation may contain binders such as gum Arabic, corn starch, microcrystalline cellulose or gelatin; excipients such as dicalcium phosphate or lactose; disintegrating agents such as alginic acid, corn starch or potato starch; lubricants such as magnesium stearate; sweeteners such as sucrose or saccharin; flavors such as peppermint, methyl salicylate or fruit flavor. In case the unit dosage form is a capsule, liquid carriers such as polyethylene glycol or fatty oil may be contained, in addition to the above-described ones. Injection for parenteral administration in the form of solution or suspension may be administered parenterally, for instance, subcutaneously, intravenously, intramuscularly, or intraperitoneally. Generally, injectable solution or suspension can be prepared by uniformly mixing an effective amount of the active ingredient with one or more pharmaceutically acceptable liquid carriers such as water, saline, aqueous dextrose and related sugar solutions, nonvolatile oil, ethanol, glycerin, glycols such as polyethylene glycol and propylene glycol. In addition, one or more auxiliaries such as antibiotics, chelating agents, buffers, preservatives, etc. may be comprised, if necessary. The above-described pharmaceutically acceptable carriers include any ones, as long as they are pharmaceutically inert and substantially nontoxic, and have no adverse effect on the activity of the active ingredient.

In addition to the above-described pharmaceutical formulations, the composition of the present invention can be manufactured into food or food supplements, by mixing it with carbonated drinks, mineral water, alcoholic beverage, chewable gum, caramels, candies, ice creams, snacks, etc., or by containing it in diet food or health supplements containing vitamins or minerals, or food additives.

The composition of the present invention may be conventionally administered at a daily dosage of 0.1˜10 mg/kg, but the dosage may be appropriately adjusted depending on age, sexuality, diet and condition of health of a patient, severity of disease, administration method, administration time, and mixing of drugs, etc.

### BEST MODE FOR CARRYING OUT THE INVENTION

Hereinafter, the present invention will be specifically described with reference to examples, but they should not be construed to limit the scope of the present invention in any manner.

### Example 1

### Preparation of a Slimming and Anti-Cellulite Composition

Using the ingredients and the contents shown in Table 1, powder of mixed rare earth elements were mixed homogeneously with other ingredients, and then, the mixture was filtered to prepare the slimming and anti-cellulite composition in the form of lotion. The prepared slimming and anti-cellulite lotion was flesh-colored and transparent, and had pH of 5.0 to 8.5.

TABLE 1
Ingredients and contents of slimming or anti-cellulite lotion

| | Component | Content (wt %) |
|---|---|---|
| 1 | Purified water | ad 100 |
| 2 | Mixed rare earth elements | 0.1 |
| 3 | Vegetable hydrogenated oil | 1.5 |
| 4 | Stearic acid | 0.6 |
| 5 | Polyglycerol-10 pentastearic&behenyl | 1.0 |
| | alcohol&sodiumstearoyl lactylate | |
| 6 | Arachidyl behenyl alcohol&arachidyl | 1.0 |
| | glucoside | |
| 7 | Cetylaryl alcohol&cetearyl glucoside | 2.0 |
| 8 | PEG-100 stearate&glycerol | 1.5 |
| | oleate&propyleneglycol | |
| 9 | Caprylic/capric triglyceride | 4.0 |
| 10 | Meadowfoam seed oil | 3.0 |
| 11 | Cetyl octanoate | 3.0 |
| 12 | Cyclomethicone | 6.0 |
| 13 | Methyl paraben | 0.2 |
| 14 | Propyl paraben | 0.1 |
| 15 | Disodium EDTA | 0.02 |
| 16 | Trimethanol amine | 0.13 |
| 17 | Glycerin | 8.0 |

### Examples 2 to 11

### Preparation of a Slimming and Anti-Cellulite Composition

A slimming and anti-cellulite lotion was prepared by substantially the same process as Example 1 above, except that La(Example 2), Ce(Example 3), Pr(Example 4), Nd(Example 5), Pm(Example 6), Sm(Example 7), Eu(Example 8), Gd(Example 9), Tb(Example 10) and Dy(Example 11) were respectively used instead of mixed rare earth elements.

### Comparative Example 1

A lotion was prepared excluding the ingredient of 2 from Table 1.

### Experimental Example 1

### Measurement of the Lipidolysis Effect of Rare Earth Elements in Adipocytes of Rats

The subcutaneous adipose tissue of a male Wistar rat was separated and minced with scissors. In 0.05% collagenase solution, it was cultivated at 37° C. for 1 hour and filtered to obtain each single adipocyte. In order to evaluate each substance for the effect of promoting degradation of neutral fat in adipocytes, the following experiment was conducted using the adipocytes that had been separated with the above-described method.

Fatty acid-free DMEM (Dulbecco's modified eagles medium) supplemented with 10% fetal bovine serum (FBS) was added to the adipocytes of 1×10⁶ per well. After 2 hours, each rare earth element or mixed rare earth elements of 10 μg were added thereto and the concentration of glycerol that was released to the medium from adipocytes was measured, to evaluate the degree of lipidolysis. The glycerol was quantified by color reaction using the GPO-Trinder kit purchased from Sigma-Aldrich (St. Louis, Mo., USA). The absorbance was measured at 540 nm using an ELISA reader. The results were shown as % of the control group. Neither rare earth elements nor mixed rare earth elements was added but only the medium was added to the control group.

The results are shown in FIG. 1. As shown in FIG. 1, it was found that the concentration of glycerol that was released to the culture medium in the mixed rare earth element treated group was remarkably increased compared to that of the control group. In addition, in the individual rare earth element treated groups, substantially the same results as that for the mixed rare earth element treated group could be obtained.

### Experimental Example 2

### Measurement of the Lipidolysis Effect of Rare Earth Elements in 3T3 L1 Cell Line

The murine preadipocytes (3T3-L1 cells, ATCC CCL-92.1) were pre-cultured in DMEM supplemented with 10% fetal bovine serum and an antibiotic in a CO₂ incubator at 37° C. for 2 days to 5×10⁴ cell/ml. Then, insulin of 2 μmol, isobutylmethylxanthine of 250 μmol, and dexamethasone of 10 μmol were added thereto to induce differentiation into adipocytes. The medium and the differentiation-inducing reagent were added thereto every two days and the cells were cultivated to examine the degree of differentiation. They were differentiated into adipocytes for 5 days or longer, and then, the medium was replaced with a new one supplemented with 10% fetal bovine serum and an antibiotic, and each 10 μg of individual rare earth elements or mixed rare earth elements were added thereto. The glycerol was quantified according to the same method as Experimental Example 1.

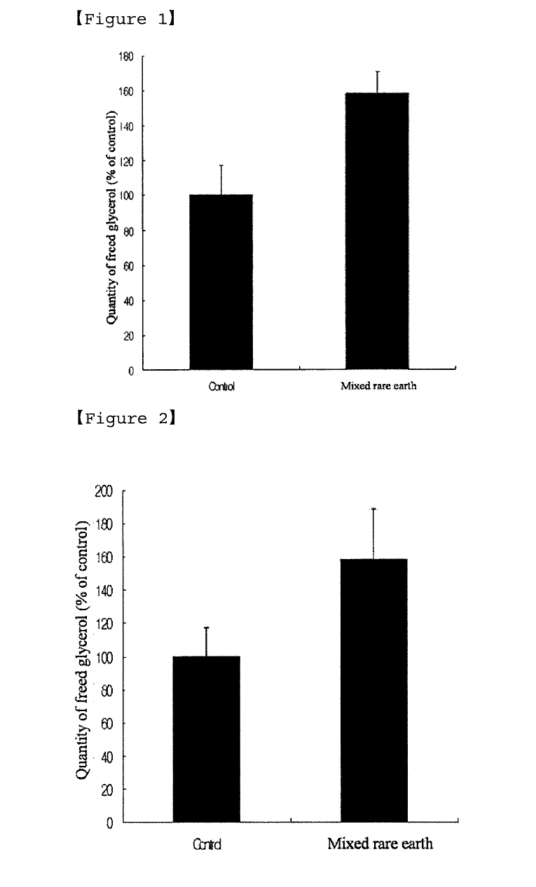

The results are shown in FIG. 2. As shown in FIG. 2, it was found that the concentration of glycerol that was released to the culture medium in the mixed rare earth element treated group was remarkably increased compared to that of the control group. In addition, in the individual rare earth element treated group, substantially the same results as that for the mixed rare earth element-treated group could be obtained.

### Experimental Example 3

### Evaluation of Skin Irritability of Rare Earth Elements

The skin irritability of rare earth elements was evaluated using a New Zealand white rabbit as follows.

A vehicle or 10% mixed rare earth elements were spread on the skin of the rabbit, twice a day, total 8 times for 4 days. Then, scores for erythema and crust formation and scores for edema formation were accumulated to get the cumulative skin irritation index. The skin cumulative irritation index was evaluated according to the criteria as shown in Table 2 below. The results are shown in Table 3. The irritation index was obtained according to the conventional method of Draize's Primary Irritation Index (P.I.I.) (see Draize, J. H., Appraisal of the safety of chemical in foods, drugs and cosmetics).

TABLE 2

| Skin irritability | | Scores |
|---|---|---|
| erythema | No erythema | 0 |
| and crust | Very weak erythema | |
| formation | (barely distinguishable with naked | 1 |
| | eyes) | |
| | Clear erythema | 2 |
| | Serious or strong erythema | 3 |
| | Dark red erythema and crust formation | 4 |
| Edema | No edema | 0 |
| formation | Very weak edema | |
| | (barely distinguishable with naked | 1 |
| | eyes) | |
| | Clear edema(clearly edged) | 2 |
| | Serious edema(swelled about 1 mm) | 3 |
| | Strong edema(swelled more than 1 mm | 4 |
| | and expanded outside the exposure | |
| | parts) | |

Primary irritation index = (averaged sum of scores for erythema and edema)/4

TABLE 3

| | Primary irritation |
|---|---|
| Ingredient | index (0~4) |
| Vehicle | 0.6 |
| Mixed rare earth | 0.8 |
| elements | |

As shown in Table 3, it was found that mixed rare earth elements have little skin irritation.

### Experimental Example 4

### Evaluation of Slimming Effect of the Composition of the Present Invention

To evaluate the slimming effect of the composition of the present invention, for 20 adult women (10 per each group) at the ages of 20's˜50's with 22˜30 Body Mass Index (BMI) and no other metabolic abnormality, the lotions obtained from the above examples and the comparative example were massaged every morning and evening, twice a day for 8 weeks. The changes for 8 weeks were examined at the time points of before, 4 weeks after, and 8 weeks after the massages of lotions had begun.

The heights and weights were measured. The height was recorded in the unit of cm using a Martin style biometer and the weight in the unit of kg using an electronic scale. To find out the % body fat of the testees, bio-electrical impedence (body fat analyzer TBF-105, Tanita, Japan) was used. The results are shown in Table 4.

TABLE 4

| | Example | | | Comparative Example | | |
|---|---|---|---|---|---|---|
| | | One month | Two months | | One month | Two months |
| | Before | after | after | Before | after | after |
| | treatment | treatment | treatment | treatment | treatment | treatment |
| weight (kg) | 63.64 ± 3.93 | 59.93 ± 3.92 | 55.30 ± 3.96 | 63.17 ± 4.64 | 62.24 ± 5.63 | 62.44 ± 4.41 |
| BMI | 25.03 ± 2.67 | 23.57 ± 2.55 | 21.75 ± 2.47 | 24.88 ± 2.58 | 24.51 ± 2.55 | 24.60 ± 2.57 |
| Body fat | 20.01 ± 2.64 | 17.14 ± 2.50 | 14.76 ± 2.38 | 19.86 ± 3.71 | 19.10 ± 3.38 | 19.34 ± 2.94 |
| mass (kg) | | | | | | |
| % Body | 31.37 ± 3.05 | 28.56 ± 3.33 | 26.62 ± 3.24 | 31.38 ± 5.19 | 30.65 ± 4.85 | 30.93 ± 4.06 |
| fat (%) | | | | | | |

BMI (kg/m²) = weight (kg)/[height(m) × height (m)]

As shown in Table 4, the composition of the present invention showed significantly higher effects for losing weight, and reducing BMI, body fat mass and % body fat, compared to that of the comparative example.

### Experimental Example 5

### Evaluation of Anti-Cellulite Effect of the Composition of the Present Invention

This experiment was conducted to find out the effect of the composition of the present invention on cellulite, in addition to the indices regarding obesity. In order to measure the thickness of subcutaneous fat in each part of the body of the testees, the testees were directed to hold their skin and subcutaneous fat with their left thumb and index finger from upside to downside, and then, the region 1 cm apart from the hand-held region was correctly clipped using a skinfold caliper (Dongwha Science DT-8 Skinfold Caliper), to read up to 0.1 mm unit. After repeating the measurements three times, the values at the time of obtaining constant values were accepted as the measured value. The measured parts were triceps, abdomen, and thigh.

triceps skinfold: middle part of the rear of shoulder in right triceps and the elbow

abdomen skinfold: 2 cm apart from the umbilicus on the right side

thigh skinfold: middle part of the front of right thigh and the knee joint

The obtained values were compared before and after the treatment and statistically analyzed. The results are shown in Table 5.

TABLE 5

| | Example | | | Comparative Example | | |
|---|---|---|---|---|---|---|
| | | One month | Two months | | One month | Two months |
| | Before | after | after | Before | after | after |
| | treatment | treatment | treatment | treatment | treatment | treatment |
| triceps | 35.24 ± 3.99 | 34.36 ± 4.03 | 30.70 ± 4.23 | 35.31 ± 3.49 | 34.60 ± 3.66 | 34.22 ± 3.43 |
| skinfold (mm) | | | | | | |
| abdomen | 39.40 ± 3.76 | 37.99 ± 3.67 | 31.57 ± 3.52 | 40.00 ± 4.03 | 38.71 ± 3.50 | 38.35 ± 3.48 |
| skinfold (mm) | | | | | | |
| thigh | 38.02 ± 3.04 | 37.00 ± 2.94 | 33.35 ± 3.35 | 37.70 ± 3.14 | 36.85 ± 3.15 | 36.34 ± 2.81 |
| skinfold (mm) | | | | | | |

As shown in Table 5, the composition of the present invention showed remarkably higher effect of eliminating cellulite than that of the comparative example.

### Experimental Example 6

### Evaluation of the Effect of the Composition of the Present Invention on Skin Firmness

In order to examine the effect of the composition of the present invention on skin firmness, visual examination was conducted by a tester. As an index of the visual examination, skin firmness was scored maximum 9 to minimum 1. The obtained values were analyzed by comparing ones before and after the treatment.

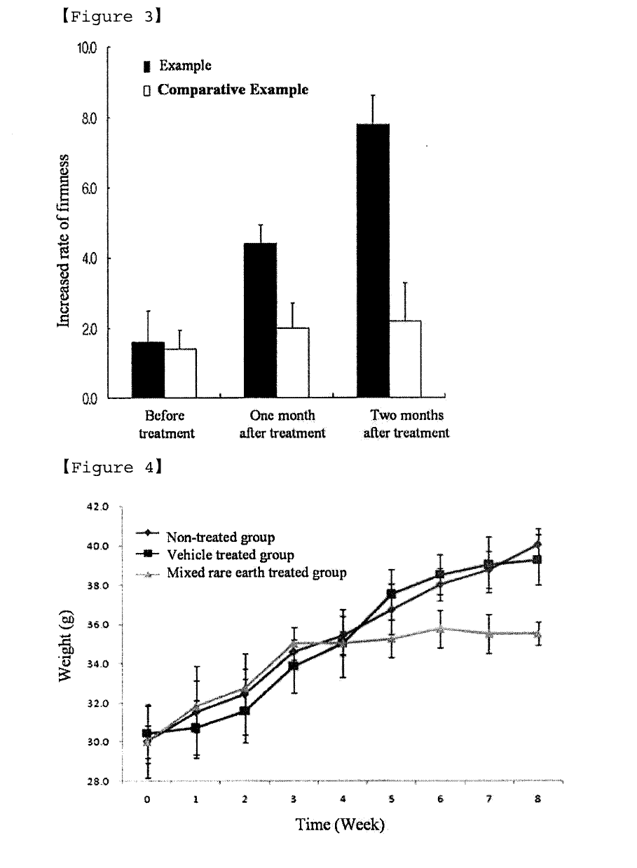

The results are shown in FIG. 3. As shown in FIG. 3, skin firmness was enhanced after the composition of the present invention had been spread.

### Experimental Example 7

### Evaluation of Skin Irritability and Side Effect of the Composition of the Present Invention in the Human Body

During the period of test, skin safety was evaluated by a dermatologist, observing skin irritation and side effects for the human body. The evaluation was performed according to the same method as Experimental Example 4, and the evaluation criteria as shown in Table 2 were used. The results are shown in Table 6.

TABLE 6

| | Primary irritation |
|---|---|
| Agent | index (0~4) |
| Vehicle | 0.6 |
| Composition of the | 0.8 |
| present invention | |

As shown in Table 6, the composition of the present invention was confirmed to have little skin irritation.

### Experimental Example 8

### Measurement of Weight Losing Effect and Toxicity of Rare Earth Elements by Oral Administration

Rare earth elements were orally administered to experimental animals to examine the weight change and survival rate. The experimental animals were 8 week-aged male ICR mice (30±1 g). Before the initiation of the experiment, they were supplied with pellets (Samyang Co.) and water ad libitum. For acclimation, they were preliminarily bred in a polypropylene box (453×293×247(H) mm, 19 l) for a week (20˜24° C., 60˜80% humidity). For oral administration, 1 mg of mixed rare earth elements were dissolved in 1 ml of triple distilled water and the solution was administered to the mice through Oral Zonde (φ 0.9×50 mm, stainless steel) at each amount of 100 mg/kg every morning and afternoon for 8 weeks.

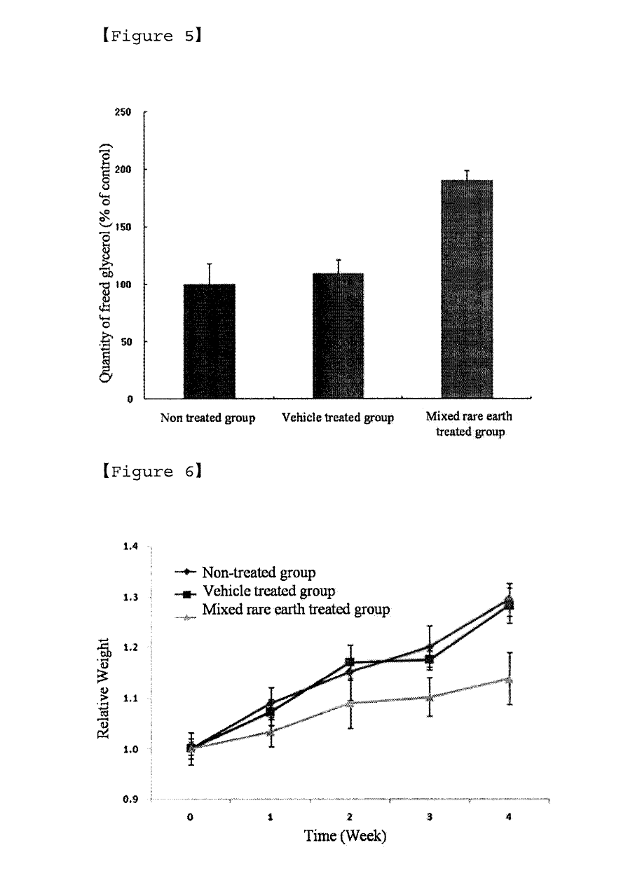

The results are shown in FIG. 4. As shown in FIG. 4, it was found that in the mixed rare earth element treated group, the weight was lost by 12.5%, compared to the non-treated and the vehicle-treated group. Therefrom, it could be concluded that mixed rare earth elements were effective for weight loss by oral administration. In addition, in the individual rare earth element-treated groups, substantially the same results as that for the mixed rare earth element-treated groups could be obtained.

During the experiment for 8 weeks, neither death nor side effect was observed, and thus, it was confirmed that rare earth elements have neither toxicity nor side effect by oral administration.

### Experimental Example 9

### Evaluation of Lipidolysis Effect of Rare Earth Elements by Oral Administration

Rare earth elements were orally administered to experimental animals to evaluate their lipidolysis effect. The blood was gathered from each experimental animal of Experimental Example 8 and only the serum was separated to quantify glycerol therein. Glycerol was quantified by color reaction using the GPO-Trinder kit purchased from Sigma-Aldrich (St. Louis, Mo., USA). The absorbance was measured at 540 nm using an ELISA reader. The results were shown as % of the control group.

The results are shown in FIG. 5. As shown in FIG. 5, it was found that in the mixed rare earth element treated group, the concentration of glycerol that was released to the serum was remarkably increased compared to the non-treated group. In addition, in the individual rare earth element treated groups, substantially the same results as that for the mixed rare earth element-treated group could be obtained.

### Experimental Example 10

### Evaluation of the Effect of Rare Earth Elements by Oral Administration on Lipid Metabolism and Obesity in the Obesity-Induced Experimental Animals

Rare earth elements were orally administered to obesity-induced experimental animals to evaluate their effect on lipid metabolism and obesity. 60 3-week old male Sprague-Dawley rats (200 g±10 g) were supplied with pellets (Samyang Co.) and water ad libitum for a week. For acclimation, they were preliminarily bred at 20˜24° C. with 60˜80% humidity, and then, obesity was induced with high fat diet for 4 weeks. The obesity inducing diet had each 5% of soybean oil (Q-one), beef tallow (Lotte Samkang), lard (Lotte Samkang), and corn oil for the fat content (20% fat as w/w), on the basis of AIN-93G (American institute of Nutrition-93G) diet. 1% cholesterol (Junsei Chemical) and casein (Daejung Chemicals & Metals Co.) were used for protein supply. For carbohydrate supply (490 carbohydrate as w/w), corn starch (Doosan Corn Products Korea) and sucrose (white sugar) were combined to induce high cholesterol type obesity. Then, they were divided into groups of ten depending on their weight using the randomized complete block design: three high fat diet maintaining groups (non-treated group, vehicle-treated group, and mixed rare earth element-treated group) and three normal pellet conversion group (non-treated group, vehicle-treated group, and mixed rare earth element-treated group). For oral administration, 1 mg of mixed rare earth elements were dissolved in 1 ml of triple distilled water and administered at each amount of 100 mg/kg every morning and afternoon for 8 weeks using Oral Zonde (φ 0.9×50 mm, Stainless Steel). After the animals had been feed with the experimental diet for 4 weeks, their weight was measured and blood was gathered. The blood was allowed to stand at 4° C. for 30 minutes and centrifuged at 3,000 rpm for 30 minutes. Then, the plasma was separated from the supernatant and kept at −70° C. for the experiment.

To evaluate the lipid concentration of the serum, the absorbance of the total cholesterol (TC, Olympus kit), HDL-cholesterol (HDL-C, Hbi kit), and triglyceride (TG, Olympus kit) were measured respectively at 555, 500, and 505 nm using Olympus AU400 (Tokyo, Japan). LDL-cholesterol (LDL-C) was calculated using the following Friedewald's formulas:

LDL-C=TC−(HDL-C+TG/5).

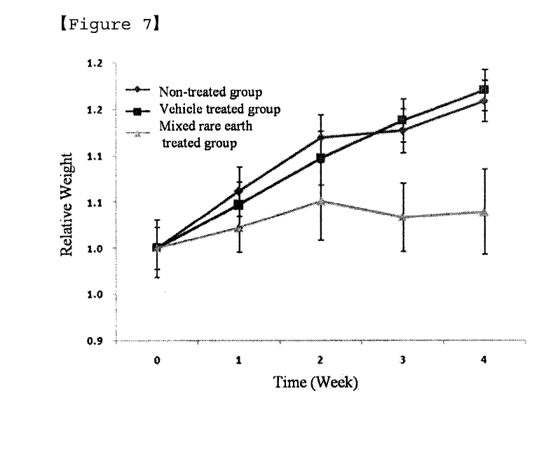

The relative weight gains for the high fat diet maintaining groups were shown in FIG. 6. As shown in FIG. 6, oral administration of mixed rare earth elements could suppress weight gain.

The results of TG, TC, HDL-C, and LDL-C in the high fat diet maintaining groups are shown in Table 7.

TABLE 7

| | TC | TG | HDL-C | LDL-C |
|---|---|---|---|---|
| | (mg/dl) | (mg/dl) | (mg/dl) | (mg/dl) |
| Non-treated | 81.87 ± 3.85 | 84.07 ± 4.55 | 24.53 ± 5.00 | 40.52 ± 0.93 |
| group | | | | |
| Vehicle- | 82.50 ± 3.11 | 83.25 ± 2.22 | 25.03 ± 4.32 | 40.82 ± 1.50 |
| treated | | | | |
| group | | | | |
| mixed rare | 67.50 ± 3.11 | 61.50 ± 03.70 | 34.50 ± 2.89 | 20.70 ± 3.62 |
| earth | | | | |
| element- | | | | |
| treated | | | | |
| group | | | | |

As shown in Table 7, mixed rare earth element-treated group had lower TC, TG, and LDL-C, while they had higher HDL-C, than the non-treated and vehicle-treated groups. From these results, it was found that the treatment with mixed rare earth elements could resolve and prevent obesity. In addition, in the individual rare earth element-treated group, substantially the same results as that for the mixed rare earth element-treated group could be obtained.

The relative weight gains for the normal pellet conversion groups were shown in FIG. 7. As shown in FIG. 7, oral administration of mixed rare earth elements after the conversion into normal pellet could suppress weight gain.

The results of TG, TC, HDL-C, and LDL-C in the normal pellet conversion groups are shown in Table 8.

TABLE 8

| | TC (mg/dl) | TG (mg/dl) | HDL-C (mg/dl) | LDL-C (mg/dl) |
|---|---|---|---|---|
| Non- | 77.00 ± 5.10 | 76.75 ± 4.65 | 24.25 ± 4.99 | 37.40 ± 9.08 |
| treated | | | | |
| group | | | | |
| Vehicle- | 75.00 ± 3.56 | 75.25 ± 4.65 | 25.75 ± 3.69 | 34.20 ± 6.90 |
| treated | | | | |
| group | | | | |
| mixed rare | 58.00 ± 4.83 | 58.00 ± 4.97 | 35.25 ± 3.59 | 11.15 ± 7.94 |
| earth | | | | |
| element- | | | | |
| treated | | | | |
| group | | | | |

As shown in Table 8, mixed rare earth element-treated group had lower TC, TG, and LDL-C, while they had higher HDL-C, than the non-treated and vehicle-treated groups. From these results, it was found that the treatment with mixed rare earth elements could resolve obesity. In addition, in the individual rare earth element-treated group, substantially the same results as that for the mixed rare earth element-treated group could be obtained.

### INDUSTRIAL APPLICABILITY

Rare earth elements promote the degradation of neutral fat accumulated in adipocytes to glycerol and free fatty acids and excretion thereof, reduce body fat to suppress obesity, reduce subcutaneous fat to make the body slim, and eliminate cellulite, which causes uneven skin, to make the skin firm and smooth, and further, have neither harm on the human body nor skin irritation.

1. A composition for promoting lipidolysis, anti-cellulite, enhancing skin firmness, or for slimming, containing, as an active ingredient, rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof.

2. The composition of claim 1, comprising 0.0001-20 weight % of the active ingredient with respect to the total weight of the composition.

3. The composition of claim 1, further comprising one or more non-toxic inert carriers.

4. The composition of claim 3, which is manufactured into hydrating toner, nutrition toner, nutrition lotion, massage cream, nutrition cream, serum, eye cream, cleansing cream, cleansing foam, cleansing water, pack, gel, spray, or powder.

5. The composition of claim 3, which is formulated into tablets, hard or soft capsules, granules, chewable tablets, pills, powder, elixir, suspension, emulsion, solution, syrup, aerosol, sachet, sterilized injectable solution or powder, lotion, ointment, gel, cream, patch or spray.

6. The composition of claim 3, which is used as a skin external formulation or a diet food.

7. A use of rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof, for promoting lipidolysis, anti-cellulite, enhancing skin firmness, or for slimming.

8. The use of claim 7, wherein the rare earth element is used by transdermal or oral administration.

9. A method for promoting lipidolysis, anti-cellulite, enhancing skin firmness, or for slimming in a subject in need thereof, comprising the step of applying to the subject rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof.

10. The method of claim 9, wherein the rare earth element is applied by transdermal or oral administration.

11. A method for preparing an agent for promoting lipidolysis, anti-cellulite, enhancing skin firmness, or for slimming, comprising using rare earth element selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), scandium (Sc) and yttrium (Y), and a mixture thereof, or a salt or an oxide thereof.
